(19) United States
(12) Patent Application Publication
(10) Pub. No.: US 2023/0158063 A1
(43) Pub. Date: May 25, 2023

(54) AIRWAY EPITHELIAL ALKALINE THERAPY TO TREAT VIRAL RESPIRATORY INFECTION

(71) Applicants: THE TRUSTEES OF INDIANA UNIVERSITY, Bloomington, IN (US); AIRBASE BREATHING COMPANY, LLC., Indianapolis, IN (US)

(72) Inventors: Michael D. DAVIS, Bloomington, IN (US); Stacey D. GILK, Bloomington, IN (US); Benjamin GASTON, Bloomington, IN (US); Christopher M. ROBINSON, Bloomington, IN (US); Diala EZZEDDINE, Indianapolis, IN (US)

(21) Appl. No.: 17/916,684

(22) PCT Filed: Apr. 6, 2021
(86) PCT No.: PCT/US21/25915

§ 371 Date: Oct. 3, 2022

Related U.S. Application Data
(60) Provisional application No. 63/005,846, filed on Apr. 6, 2020.
(60) Provisional application No. 63/019,060, filed on May 1, 2020.
(60) Provisional application No. 63/056,136, filed on Jul. 24, 2020.
(60) Provisional application No. 63/117,623, filed on Nov. 24, 2020.

(51) Int. Cl. A61K 33/00; A61K 9/08; A61K 9/00; A61K 31/675; A61K 31/245; A61K 47/02; A61P 31/14

(52) U.S. Cl. CPC A61K 33/00; A61K 9/08; A61K 9/0078; A61K 31/675; A61K 31/245; A61K 47/02; A61P 31/14

(57) ABSTRACT

The present disclosure relates to a method of treating or preventing a viral infection by raising the pH of the airways of a subject. The effect can be mediated directly by administering a pharmaceutically acceptable basic solution. Additionally, the method provides co-administering an anti-viral composition to the subject.

### CROSS REFERENCE TO RELATED APPLICATIONS

This application claims priority to U.S. Provisional Application 63/005,846, filed Apr. 6, 2020; U.S. Provisional Application 63/019,060, filed May 1, 2020; U.S. Provisional Application 63/056,136, filed Jul. 24, 2020; and U.S. Provisional Application 63/117,623, filed Nov. 24, 2020, the contents of each is incorporated herein by reference.

### GOVERNMENT SUPPORT CLAUSE

This invention was made with government support under HL128192 awarded by National Institutes of Health. The government has certain rights in the invention.

### BACKGROUND

The present disclosure relates generally to a method of treating or preventing a respiratory infection in a subject. More specifically, the present disclosure relates to administering a first composition capable of alkalinizing the airway of a subject.

Patients admitted to the hospital with COVID-19 generally have pneumonia and abnormal chest imaging. Complications include acute respiratory failure, acute respiratory distress syndrome (ARDS), and acute myocardial injury. ARDS appears to be a significant predictor of patient mortality. Death typically results from severe respiratory infection leading to ARDS. Both the illness severity and infection course are more severe in the elderly and in individuals with underlying comorbidities, including cardiovascular and chronic respiratory diseases.

Using the coronavirus spike (S) protein, the SARS-CoV-2 virus engages the angiotensin-converting enzyme 2 (ACE2) receptor as a means to gain entry into human cells. This receptor is highly expressed in the airway epithelium, among other cell types and organs. To bind to cell membranes, SARS-CoV-2 requires S protein cleavage either by the transmembrane protease serine 2 (TMPRSS2) or by the cathepsin B and L (CatB/L) endosomal complex.

Specifically, TMPRSS2 can be inhibited by camostat methylate (CM), but CM is an irritant and may not be ideal for airway administration in patients with evolving ARDS. CatB/L is inhibited by endosomal alkalinization using ammonium chloride.

There exists a need to provide a non-invasive method of treatment for respiratory infections, especially in light of the most recent COVID-19 pandemic.

### SUMMARY

Intracellular alkalinization was able to block SARS-CoV-2 entry into primary human airway epithelial cells, evaluated in human airway epithelial cell alkalinization in vitro. Inhalation of solution is safe, alkalinizes the airway intracellular space and, in so doing, inhibits CatB/L to prevent viral entry in the human airway epithelium. Accordingly, based on this discovery, several novel therapeutic strategies for the prevention and treatment of viral respiratory infections can be employed including: 1) prophylactically administering an aerosolized solution to a subject to prevent a viral respiratory infection, 2) administering an aerosolized solution to treat a subject suspected of having a viral respiratory infection, 3) administering an aerosolized solution to treat a symptom of a viral respiratory infection in a subject, and 4) administering an aerosolized solution and administering an antiviral composition to a subject to treat a symptom of or to treat a subject suspected of having a viral respiratory infection.

In some embodiments, a method of treating a patient with COVID-19 comprises administering an effective amount of a solution. In some embodiments, the solution comprises an alkaline glycine buffer. In some embodiments, the method further comprises administering an effective amount of remdesivir.

The following additional embodiments are also contemplated and are within the scope of this disclosure.

Clause 1. A method of treating a viral respiratory infection caused by a virus comprising: contacting the airways of a subject with a solution, the solution comprising an alkalinized buffer, wherein the solution has a pH ranging from about 7.4 to about 11.0, and wherein the solution is formulated as an aerosol.

Clause 2. The method of clause 1, wherein the solution inhibits the CatL/B pathway in the cells in the airways of the subject and reduces the viral infection rate.

Clause 3. The method of clause 1, wherein the cells include epithelial cells.

Clause 4. The method of clause 1, wherein the alkalinized buffer comprises one or more of a buffer, a titrating base, and an osmotic balancing agent.

Clause 5. The method of clause 4, wherein the alkalinized buffer comprises a buffer, a titrating base, and an osmotic balancing agent.

Clause 6. The method of clauses 4 or 5, wherein the buffer comprises an amino acid.

Clause 7. The method of clause 6, wherein the amino acid is selected from glycine, alanine, valine, leucine, isoleucine, methionine, tryptophan, asparagine, glutamine, serine, threonine, tyrosine, cysteine, aspartic acid, glutamic acid, arginine, or histidine.

Clause 8. The method of clause 6, wherein the buffer comprises at least two amino acids.

Clause 9. The method of clause 8, wherein the ratio of the at least two amino acids are present at about 10:1, 9:1, 8:1, 7:1, 6:1, 5:1, 4:1, 3:1, 3:2, 2:1, 1:1, 2:3, 1:2, 1:3, 1:4, 1:5, 1:6, 1:7, 1:8, 1:9, or 1:10.

Clause 10. The method of clauses 4 or 5, wherein the buffer is present at a concentration of about 50 mM to about 250 mM.

Clause 11. The method of clauses 4 or 5, wherein the buffer has a pH ranging from about 8 to about 11.5.

Clause 12. The methods of clauses 4 or 5, wherein the titrating base comprises a hydroxide.

Clause 13. The methods of clause 12, wherein the titrating base comprises sodium hydroxide.

Clause 14. The method of clause 13, wherein the titrating base is sodium hydroxide.

Clause 15. The method of clauses 4 or 5, wherein the titrating base is present at a concentration of about 25 mM to about 150 mM.

Clause 16. The method of clauses 4 or 5, wherein the titrating base comprises two components.

Clause 17. The method of clause 16, wherein the two components of the titrating base are present in a ratio of about 10:1, 9:1, 8:1, 7:1, 6:1, 5:1, 4:1, 3:1, 3:2, 2:1, 1:1, 2:3, 1:2, 1:3, 1:4, 1:5, 1:6, 1:7, 1:8, 1:9, or 1:10.

Clause 18. The method of clauses 4 or 5, wherein the titrating base has a pH ranging from about 8 to about 14.

Clause 19. The method of clauses 4 or 5, wherein the osmotic balancing agent comprises sodium.

Clause 20. The method of clause 19, wherein the osmotic balancing agent comprises sodium chloride.

Clause 21. The method of claim 20, wherein the osmotic balancing agent is sodium chloride.

Clause 22. The method of clause 21, wherein the osmotic balancing agent comprises two components.

Clause 23. The method of clause 22, wherein the two components of the osmotic balancing agent are present in a ratio of about 10:1, 9:1, 8:1, 7:1, 6:1, 5:1, 4:1, 3:1, 3:2, 2:1, 1:1, 2:3, 1:2, 1:3, 1:4, 1:5, 1:6, 1:7, 1:8, 1:9, or 1:10.

Clause 24. The method of clauses 4 or 5, wherein the osmotic balancing agent has a pH ranging from about 5.5 to about 8.0.

Clause 25. The method of clause 1, wherein the solution further comprises water.

Clause 26. The method of clause 25, wherein the water is present in an amount ranging from 60% to about 99.9% by volume of the total solution volume.

Clause 27. The method of clause 1, wherein the solution further comprises a phosphate buffer solution.

Clause 28. The method of clauses 25-28, wherein the water or phosphate buffer has a pH ranging from about 7 to about 11.5.

Clause 29. The method of clause 1, wherein the alkalinized buffer has a pH ranging from about 8 to about 11.5.

Clause 30. The method of clause 1, wherein the solution has a pH ranging from about 8 to about 11.5 or about 9.4 to about 9.8.

Clause 31. The method of clause 1, wherein the solution is delivered at a temperature ranging from about 18° C. to about 38° C.

Clause 32. The method of clause 1, wherein the solution is formulated as an aerosol.

Clause 33. The method of clause 32, wherein the aerosol is delivered using a nebulizer, squeeze bottle, or solution atomizer.

Clause 34. The method of clause 33, wherein the nebulizer is selected from an ultrasonic nebulizer, conventional nebulizer, or vibrating mesh nebulizer.

Clause 35. The method of clauses 1-33, wherein the solution is present in an amount ranging about 0.25 mL to about 10 mL.

Clause 36. The method of clause 10, wherein the buffer is present in the solution at a concentration of about 100 mM to about 150 mM.

Clause 37. The method of clause 36, wherein the buffer is present in the solution at a concentration of about 120 mM.

Clause 38. The method of clause 1, further comprising administering an antiviral drug to the airway.

Clause 39. The method of clause 38, wherein the antiviral drug comprises a viral RNA polymerase inhibitor.

Clause 40. The method of clause 39, wherein the viral RNA polymerase inhibitor is remdesivir.

Clause 41. The method of clause 40, wherein remdesivir is administered by injection.

Clause 42. The method of clause 38 wherein the antiviral is administered close in time to solution.

Clause 43. The method of claim 38, wherein the antiviral is administered before administering the solution.

Clause 44. The method of claim 38, wherein the antiviral is administered after administering the solution.

Clause 45. The method of clause 1, further comprising administering a serine protease inhibitor.

Clause 46. The method of claim 45, wherein the serine protease inhibitor comprises camostat methylate.

Clause 47. The method of clause 2, wherein the pH of the cells raises to between about 8.0 to about 10.5.

Clause 48. The method of clause 1, wherein the viral infection rate is reduced compared to a non-treated subject.

Clause 49. A method treating a viral respiratory infection in a subject comprising, administering to the subject an effective amount a solution as described in claims 1-48.

Clause 50. The method of clause 49, wherein the viral respiratory infection is caused by a virus.

Clause 51. The method of clause 50, wherein the virus is selected from coronaviruses, rhinoviruses, respiratory syncytial viruses, influenza viruses, picornaviruses, alphaviruses, flaviviruses, retroviruses, or a combination thereof.

Clause 52. The method of clause 51, wherein the coronavirus is selected from SARS-CoV-2, MERS-CoV, or SARS, or mutated forms thereof.

Clause 53. The method of clause 52, wherein the subject has been diagnosed with COVID-19.

Clause 54. The method of clause 49, wherein the solution is formulated to be administered as an aerosol.

Clause 55. The method of clause 54, wherein the aerosol is delivered by a nebulizer or nasal spray.

Clause 56. The method of clause 49, wherein the effective amount of the buffer is administered in an amount ranging from about 100 mM to about 200 mM present in solution.

Clause 57. The method of clause 49, wherein the solution is administered in a unit dose.

Clause 58. The method of clause 57, wherein a dose comprises about 0.50 mL of solution to about 10 mL of solution.

Clause 59. The method of clause 58, wherein the dose comprises about 15 mg to about 30 mg of buffer.

Clause 60. The method of clause 57, wherein the dose comprises about 22.5 mg of buffer.

Clause 61. The method of clause 57, wherein the dose is administered by nasal spray, squeeze bottle, nasal nebulizer, or by nasal mucosal atomization device.

Clause 62. The method of clause 57, wherein the subject is dosed once a day

Clause 63. The method of clause 57, wherein the subject is dosed at least twice a day.

Clause 64. The method of clause 57, wherein the subject is dosed up to three times a day.

Clause 65. The method of clause 49, wherein the solution is administered to the subject for at least one week.

Clause 66. The method of clause 49, wherein the solution is administered to the patient for up to eight weeks.

Clause 67. The method of clause 49, wherein the solution is administered for about one week, about two weeks, about three weeks, about four weeks, about five weeks, about six weeks, about seven weeks, or about eight weeks.

Clause 68. The method of clause 49, further comprising administering an antiviral.

Clause 69. The method of clause 68, wherein the antiviral is co-administered.

Clause 70. The method of clause 68, wherein the antiviral is administered after administration of the solution.

Clause 71. The method of clause 68, wherein the antiviral is administered before administration of the solution.

Clause 72. The method of clause 68, wherein the antiviral is administered to the subject by injection.

Clause 73. The method of clause 68, wherein the antiviral formulated to be administered in an aerosolized form.

Clause 74. The method of clause 49, wherein the solution further comprises a composition configured to provide a pleasant scent.

Clause 75. The method of clause 74, wherein the composition is an essential oil.

Clause 76. The method clause 74, wherein the composition is an herb, flower, or perfume.

Clause 77. The method of claim 57, wherein the dose comprises a concentration of about 17 mM to about 300 mM or 1.53 mg/mL to about 27 mg/mL of the buffer in the solution.

Unless otherwise specifically noted, all recited percentages refer to a percent by volume.

### BRIEF DESCRIPTION OF THE DRAWINGS

The following description accompanies the drawings, all given by way of non-limiting examples that may be useful to understand how the described method and kit may be embodied.

FIG. 1 shows four graphs and the optical response of human epithelial cells in response to control, zinc chloride (ZnCl²), or an exemplary formulation of the solution;

FIG. 2 is a graph showing the plaque forming units of SARS-CoV-2 in the control sample and a treated sample with an exemplary embodiment of the solution;

FIG. 3 is a graph comparing the limit of detection of control HAE cells to treated/pretreated HAE cells with an exemplary embodiment of the solution in 24 hour increments;

FIG. 4 is a graph showing the ratio of Nucleocapsid viral protein to Beta-Actin protein in the HAE cells comparing control to treated cells; and

FIG. 5 is a western blot quantified in FIG. 4.

### DETAILED DESCRIPTION

A method for treating a subject having a viral respiratory infection and a kit are provided. In some embodiments, a method for reducing viral infection rate of a cell is provided. In some embodiments, a method of reducing a symptom of COVID-19 is provided. In some embodiments, a method of preventing a SARS-CoV-2 viral infection rate is provided. In some embodiments, a method of treating a subject suspected of having a viral infection is disclosed. Each method discloses a solution, or pharmaceutical formulation thereof, wherein the solution comprises an alkalinized buffer.

In an illustrative embodiment, a method of treating the symptoms associated with a viral respiratory infection in a subject is provided. The method comprises contacting the airways of a subject with a solution, wherein the solution comprises an alkalinized buffer. In some embodiments, the solution has a pH ranging from about 7.4 to about 11.0. In some embodiments, the solution is capable of raising the intracellular pH of a subject's airway cells to about 8.0 to about 11.0, about 8.5 to about 10.5, or about 9.0 to about 10.0.

In some embodiments, a method of preventing viral infection rate of airway cells is provided. The method comprises administering or delivering a solution comprising an alkalinized buffer to the cells, wherein the solution raises the intracellular pH of the cells and reduces the viral infection rate. In some embodiments, the virus causing the viral infection may be SARS-CoV-2.

In an illustrative embodiment, a method of treating a subject suspected of having a respiratory viral infection is provided. The method comprises administering to the subject a solution comprising an alkalinized buffer.

In an illustrative embodiment, a method of preventing a viral respiratory infection in a subject is provided. The method comprises administering a solution prior to the subject's exposure to a virus, wherein the solution comprises an alkalinized buffer.

The human airways or “human airway system” may be characterized as the upper airway and lower airway. The upper airway includes nasal cavities, pharynx, and larynx, whereas the lower airway includes the trachea, primary bronchi, and lungs. In some embodiments, the solution contacts the upper airway. In some embodiments, the solution contacts, the upper airway and the lower airway. In some embodiments, the solution contacts the upper or lower airway. In some embodiments, the solution is targeted to the lower airway. Targeting of the lower airway may be achieved using a solution atomizer.

The human airway system can also be characterized by the cell types within these structures. The human airways are covered with a continuous epithelial sheet. The airway epithelium is pseudostratified in the large airways and then columnar and cuboidal in the smaller airways. The epithelial cells of the human airway include ciliated, columnar, undifferentiated, secretory, and basal cells. The human airway also includes cartilage cells, Clara secretory cells, mucus glands, and neuroendocrine cells. As the airway epithelium merges with the alveolar epithelium, type I and type II cells are introduced.

In some embodiments, the solution contacts a cell in the human airway system. In some embodiments, the cell is selected from an epithelial cell, a cartilage cell, a Clara cell, a type I cell, a type II cell, a mucus gland cell, or a neuroendocrine cell. In some embodiments, the solution contacts an epithelium cell selected from the group consisting of ciliated, columnar, undifferentiated, secretory, and basal. In some embodiments, the cell is an epithelial cell. In some embodiments, the cell is an airway epithelial cell.

In some embodiments, the method provides contacting the airway of a subject with a solution. In some embodiments, the method provides administering or delivering a solution to a subject to prevent or treat a viral respiratory infection. In an illustrative aspect, the solution comprises an alkalinized buffer. The solution components may be individualized to the subject depending on results from a non-invasive measurements of airway redox chemistry. For example, measurements of a subject's airway pH may be taken and analyzed using condensation techniques. Condensation techniques, including exhaled breath condensation techniques are known to those skilled in the art.

In some embodiments, the alkalinized buffer comprises one or more of a buffer, a titrating base, and an osmotic balancing agent. In some embodiments, the alkalinized buffer comprises a buffer, a titrating base, and an osmotic balancing agent. In some embodiments, the alkalinized buffer consists essentially of a buffer, a titrating base, and an osmotic balancing agent.

In some embodiments, the buffer comprises an amino acid. In some embodiments, the amino acid is selected from glycine, alanine, valine, leucine, isoleucine, methionine, tryptophan, asparagine, glutamine, serine, threonine, tyrosine, cysteine, aspartic acid, glutamic acid, arginine, or histidine. In some embodiments, the buffer comprises more than one amino acid. In some embodiments, the buffer comprises glycine (aminoacetic acid), bicine (N,N-Bis(2-hydroxyethyl)glycine, tricene (N-[tris(hydroxymethyl)methyl]glycine, CAPAS (3-(Cyclohexamino)-1-propanesulphonic acid, CAPSO (3-(Cyclohexamino)-2-hydroxypropanesulphonic acid, 2-(Cyclohexamino)-ethenesulphonic acid), phosphate, tris (hydroxylmethyl) aminomethane (THAM) or a combination thereof.

In some embodiments, the buffer comprises glycine. In some embodiments, the buffer comprises glutamine. In some embodiments, the buffer consists essentially of glycine.

In some embodiments, wherein the buffer comprises two amino acids, the ratio of the two amino acids may be present at about 10:1, 9:1, 8:1, 7:1, 6:1, 5:1, 4:1, 3:1, 3:2, 2:1, 1:1, 1:2, 1:3, 1:4, 1:5, 1:6, 1:7, 1:8, 1:9, or 1:10.

In some embodiments, the buffer is present in the solution at a concentration ranging from about 17 mM to about 300 mM, about 50 mM to about 250 mM, about 100 mM to about 200 mM, or about 125 mM to about 175 mM. In some embodiments, the buffer is present at a concentration of at least about 50 mM, at least about 60 mM, at least about 70 mM, at least about 80 mM, at least about 90 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, or at least about 200 mM. In some embodiments, the buffer is present at a concentration of less than about 200 mM, less than about 175 mM, less than about 150 mM, less than about 125 mM, or less than about 100 mM. In some embodiments, the buffer is present at a concentration ranging from about 100 mM to about 150 mM, about 100 mM to about 140 mM, about 100 mM to about 130 mM, about 110 mM to about 130 mM, about 115 mM to about 125 mM, or about 118 mM to about 122 mM. In some embodiments, the buffer is present at a concentration of about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, or about 130 mM. In some embodiments, the buffer is present at a concentration of about 120 mM. In some embodiments, the buffer is present at a concentration of 120 mM.

In some embodiments, the buffer has a pH ranging from about 7.4 to about 11.5. In some embodiments, the buffer has a pH ranging from about 7.5 to about 11.0, from about 8.0 to about 10.5, from about 8.5 to about 10, or from about 9.0 to about 9.5. In some embodiments, the buffer has a pH of at least about 7.5, at least about 8.0, at least about 8.5, at least about 9.0, at least about 9.5, at least about 10.0, at least about 10.5, at least about 11.0, or at least about 11.5. In some embodiments, the buffer has a pH of less than about 11.5, less than about 11.0, less than about 10.5, less than about 10.0, less than about 9.5, less than about 9.0, less than about 8.5, or less than about 8.0. In some embodiments, the buffer has a pH of about 7.5, about 8.0, about 8.5, about 9.0, about 9.5, about 10.0, about 10.5, about 11.0, or about 11.5. In some embodiments, the buffer has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5 about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, or about 11.5.

In some embodiments, the titrating base comprises a hydroxide. In some embodiments, the titrating base comprises sodium hydroxide. In some embodiments, the titrating base is sodium hydroxide. In some embodiments, the titrating base comprises more than one component.

In some embodiments, wherein the titrating base comprise two components, the two components are present in the titrating base in a ratio of about 10:1, 9:1, 8:1, 7:1, 6:1, 5:1, 4:1, 3:1, 3:2, 2:1, 1:1, 2:3, 1:2, 1:3, 1:4, 1:5, 1:6, 1:7, 1:8, 1:9, or 1:10.

In some embodiments, the titrating base is present at a concentration ranging from about 25 mM to about 150 mM. In some embodiments, the titrating base is present at a concentration of about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM. In some embodiment, the titrating base is present at a concentration at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, the titrating base is present at a concentration of about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, or about 75 mM. In some embodiments, the titrating base is present at a concentration of about 70 mM. In some embodiments, the titrating base is present at a concentration of 70 mM.

In some embodiments, the titrating base has a pH ranging from about 9 to about 14 or about 7.4 to about 11.5. In some embodiments, the titrating base has a pH ranging from about 7.5 to about 11.0, from about 8.0 to about 10.5, from about 8.5 to about 10, or from about 9.0 to about 9.5. In some embodiments, the titrating base has a pH of at least about 7.5, at least about 8.0, at least about 8.5, at least about 9.0, at least about 9.5, at least about 10.0, at least about 10.5, at least about 11.0, at least about 11.5, at least about 12.0, at least about 12.5, at least about 13.0, at least about 13.5, or at least about 14.0. In some embodiments, the titrating base has a pH of less than about 11.5, less than about 11.0, less than about 10.5, less than about 10.0, less than about 9.5, less than about 9.0, less than about 8.5, or less than about 8.0. In some embodiments, the titrating base has a pH of about 7.5, about 8.0, about 8.5, about 9.0, about 9.5, about 10.0, about 10.5, about 11.0, about 11.5, about 12.0, about 12.5, about 13.0, about 13.5, or about 14.0. In some embodiments, the titrating base has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5 about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, or about 11.5.

In some embodiments, the osmotic balancing agent comprises sodium. In some embodiments, the osmotic balancing agent comprises sodium chloride. In some embodiments, the osmotic balancing agent is sodium chloride. In some embodiments, the osmotic balancing agent comprises more than one component. In some embodiments wherein the osmotic balancing agent comprises two components, the two components of the osmotic balancing agent is present in a ratio of about 10:1, 9:1, 8:1, 7:1, 6:1, 5:1, 4:1, 3:1, 3:2, 2:1, 1:1, 2:3, 1:2, 1:3, 1:4, 1:5, 1:6, 1:7, 1:8, 1:9, or 1:10.

In some embodiments, the osmotic balancing agent is present in the solution at a concentration ranging from about 50 mM to about 200 mM, about 75 mM to about 175 mM, about 100 mM to about 150 mM, or about 125 mM to about 130 mM. In some embodiments, the osmotic balancing agent is present at a concentration of about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, or about 110 mM. In some embodiments, the osmotic balancing agent is present in the solution at a concentration of about 102 mM. In some embodiments, the osmotic balancing solution is present at a concentration of 102 mM.

In some embodiments, the osmotic balancing agent has a pH ranging 5.5 to about 8.0. In some embodiments, the osmotic balancing agent has a pH ranging from about 6.0 to about 7.5, or from about 6.5 to about 7.0. In some embodiments, the osmotic balancing agent has a pH of at least about 5.5, at least about 6.0, at least about 6.5, at least about 7.0, at least about 7.5, or at least about 8.0. In some embodiments, the osmotic balancing agent has a pH of less than about 11.5, less than about 11.0, less than about 10.5, less than about 10.0, less than about 9.5, less than about 9.0, less than about 8.5, or less than about 8.0. In some embodiments, the osmotic balancing agent has a pH of about 5.5, about 6.0, about 6.5, about 7.0, about 7.5, or about 8.0. In some embodiments, the osmotic balancing agent has a pH of about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0 about 7.1, about 7.2, about 7.3, about 7.4, about 7.5, about 7.6, about 7.7, about 7.8, about 7.9, or about 8.0.

In an illustrative embodiment, the alkalinize buffer comprises a buffer, a titrating base, and an osmotic balancing agent. In some embodiments, the buffer comprises glycine. In some embodiments, the buffer is glycine. In some embodiments, the titrating base comprises sodium hydroxide. In some embodiments, the titrating base is sodium hydroxide. In some embodiments, the osmotic balancing agent comprises sodium chloride. In some embodiments, the osmotic balancing agent is sodium chloride. In some embodiments, the alkalinizing buffer comprises glycine, sodium hydroxide, and an osmotic balancing agent. In some embodiments, the alkalinizing buffer comprises glycine, a titrating base, and sodium chloride. In some embodiments, the alkalinized buffer comprises a buffer, sodium hydroxide, and sodium chloride. In some embodiments, the alkalinized buffer comprises glycine, sodium hydroxide, and sodium chloride. In some embodiments, alkalinized buffer comprises glycine at a concentration of about 120 mM, sodium hydroxide at a concentration of about 70 mM, and sodium chloride at a concentration of about 102 mM.

In some embodiments, the buffer, titrating base, and osmotic balancing agent are present in the alkalinized buffer in a ratio of about 1:1:1, 2:1:1, 2:1:2, 3:1:1, or 3:1:2.

In some embodiments, the alkalinized buffer has a pH ranging from about 8.0 to about 12.0, about 8.0 to about 11.5, about 8.0 to about 10.0, about 8.5 to about 11.0, or about 9.0 to about 10.5. In some embodiments, the alkalinized buffer has a pH of about 8.0, about 8.5, about 9.0, about 9.5, about 10.0, about 10.5, about 11.0, or about 11.5. In some embodiments, the alkalinized buffer has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0. In some embodiments, the alkalinized buffer and the solution have the same pH. In some embodiments, the solution has a different pH than the alkalinized buffer isolated.

In some embodiments, the alkalinized buffer further comprises an aqueous solution. In some embodiments the solution comprises water. In some embodiments, the water may be purified and/or sterilized. For example, the water may be subjected to deionization (e.g., capacitive deionization or electrodeionization), reverse osmosis, carbon filtering, microfiltration, ultrafiltration, and/or ultraviolet sterilization. In some embodiments, the water is deionized. In some embodiments, the water is of a quality known as “sterile water” suitable for inhalation.” Water for inhalation is generally made by distillation or reverse osmosis. Water for inhalation is a sterile, nonpyrogenic, solute-free preparation of water, chemically designated “H₂O,” and having a pH of between about 5.5 and about 7.5. In some embodiments, the solution is produced using commercially purchased saline solution of a type and concentration suitable for inhalation. In some embodiments, the alkalinized buffer further comprises phosphate buffer solution.

In some embodiments, the water or saline solution is used to formulate an aerosolized alkalinized buffer. In some embodiments, the water or saline solution is present in an amount ranging from about 50% to about 99.99% by volume of the total solution volume. In some embodiments, the water or saline solution is present in an amount of at least about 50%, about 55%, about 60%, about 65%, about 70%, about 75%, about 80%, about 85%, about 90%, about 95%, or about 99.99% by volume of total solution volume. In some embodiments, the water or saline solution is present in an amount of about 90%, about 91%, about 92%, about 93%, about 94%, about 95%, about 96%, about 97%, about 98%, or about 99% by volume of total solution volume. In some embodiments, the water or saline solution is present in an amount ranging from about 85% to about 99.99%, from about 90% to about 99.99%, from about 92% to about 99%, from about 95% to about 98% by volume of total solution volume.

In some embodiments, wherein a saline solution is used, the solution does not include an osmotic balancing agent.

In an illustrative embodiment, the solution comprises an alkalinized buffer. In some embodiment, the solution has a pH ranging from about 8 to about 11.5, about 8.5 to about 11.0, about 9.0 to about 10.5, or about 9.5 to about 10.0. In some embodiments, the solution has a pH ranging from about 9.4 to about 9.8. In some embodiments, the solution has a pH of about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, or about 10.5. In some embodiments, the solution has a pH of 9.0, 9.1, 9.2, 9.3, 9.4, 9.5, 9.6, 9.7, 9.8, 9.9, 10.0, 10.1, 10.2, 10.3, 10.4, or 10.5.

In an illustrative embodiment, the solution is formulated to be an aerosol. In some embodiments, the solution is not provided as a colloid. In some embodiments, the aerosol is delivered or administered by a delivery device. In some embodiments, the delivery device is a nebulizer, such as, an ultrasonic nebulizer, a vibrating mesh nebulizer, or a conventional nebulizer. In some embodiments, the delivery device is a nasal spray bottle, a squeeze bottle, a nasal nebulizer, or by a solution atomizer such as a nasal mucosal atomization device. In some embodiments, the solution is provided as a mist, spray, or atomization, which are all considered here to be an aerosol. In some embodiments, the solution is specifically not provided as a vapor.

In some embodiments, conventional nebulizations may be administered or delivered using a commercially-available nebulizer with an output mass median aerodynamic diameter (mmad) of about 0.1 mmad to about 12 mmad, or about 2 mmad to about 10 mmad. For more distal airway targeting, an ultrasonic nebulizer or solution atomizer may be used; for more proximal, a more conventional HFA- or dry-powdered delivery may be used. In some embodiments, more distal airway infections would require a smaller particle nebulizer; more severe infections or more severe airway inflammation could require a higher pH or more frequent dosing.

In some embodiments, the aerosol is provided with an output mass median aerodynamic diameter of about 0.1, about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, or about 12.0 mmad.

In some embodiments, the method further comprises administering or delivering an anti-viral composition to the subject. In some embodiments, the anti-viral composition includes a viral RNA polymerase inhibitor. In some embodiments, the anti-viral composition comprises remdesivir. In some embodiments, the anti-viral composition is remdesivir. In some embodiments, the anti-viral composition is selected from a broad-spectrum antiviral drug, or an anti-viral drug formulated to target an influenza virus, a coronavirus, a rhinovirus, a respiratory syncytial virus, a picornavirus, an alphavirus, a flavivirus, or a retrovirus. In some embodiments, the anti-viral composition is administered in the form of an injection. In some embodiments, the anti-viral composition is administered or delivered in the form of an oral formulation. In some embodiments, the anti-viral composition is administered or delivered in the form of a powder or aerosol that may be delivered by the delivery device.

In some embodiments, the method further comprises administering or delivering a serine protease inhibitor. A serine protease inhibitor is characterized by the presence of three critical amino acids—histidine, aspartate, and serine—in the catalytic site. In some embodiments, the serine protease inhibitor comprises camostat methylate. In some embodiments, the serine protease inhibitor is camostat methylate. In some embodiments, the serine protease inhibitor is administered or delivered by way of, such as, injection, oral formulation, or aerosol or powder using the delivery device.

In an illustrative aspect, the method comprises contacting the airways of a subject with a solution, wherein the solution comprises an alkalinized buffer. In some embodiments, the solution is delivered to the airways as an aerosol. In some embodiments, the step of delivering is performed by a delivery device.

In an illustrative aspect the method comprises administering a solution to a subject suspected of having COVID-19. In some embodiments, the method further comprises administering an anti-viral composition. In some embodiments, the method comprises administering a solution to a subject to treat a symptom of a viral respiratory infection, wherein the infection is caused, in whole or in part by, coronaviruses, rhinoviruses, respiratory syncytial viruses, influenza viruses, picornaviruses, alphaviruses, flaviviruses, or retroviruses. In some embodiments, the coronavirus is selected from SARS-CoV-2, MERS-CoV, or SARS, mutated forms of these coronaviruses, or a combination thereof. In some embodiments, the coronavirus is SARS-CoV-2.

The term administered refers to administration of a component, such as the solution, antiviral, or serine protease inhibitor, by a medical professional including a doctor, registered nurse practitioner, nurse, pharmacist, pharmacist technician, and the like who are licensed or otherwise authorized to administer a component to a subject.

The term delivered refers to solution traveling into the airway by a device that generates an aerosol.

In some embodiments, the solution is administered or delivered to the airway at temperature ranging from about 18° C. to about 40° C. In some embodiments, the solution is administered or delivered to the airway at ambient temperature. Ambient temperature is defined as the average temperature of a given place or space. In some embodiments, the solution is administered or delivered to the airway at a temperature of about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In carrying out the methods of this disclosure, the amount of alkalinized buffer in the solution is adequate to achieve a therapeutic effect. As used herein, the term “therapeutically effective amount” refers to an amount which gives the desired benefit to a subject and includes both treatment and prophylactic administration. The amount will vary from one subject to another and will depend upon a number of factors, including the overall physical condition of the subject and the underlying cause of the condition to be treated.

The amount of alkalinized buffer used for treating or preventing a respiratory viral infection, relieving one symptom of a respiratory viral infection, or reducing viral entry into the subject's airway gives an acceptable rate of change and maintains desired response at a beneficial level. A therapeutically effective amount of the alkalinized buffer used in the methods of the present disclosure may be readily ascertained by one of ordinary skill in the art using publicly available materials and procedures. In one embodiment of the present methods, the therapeutically effective amount of alkalinized buffer to be delivered can be quantified by determining milligrams of alkalinized buffer per mL of overall solution, milligrams of the buffer to be delivered in a dose, or concentration of buffer present in the alkalinized buffer.

In various aspects of the method, the therapeutically effective amount is an amount sufficient to achieve a desired pH level of the airway fluid lining and/or intracellular pH of at least one cell in a subject's airway.

In some embodiments, the solution is administered or delivered as a dose. In some embodiments, the dose is an amount ranging about 0.25 mL to about 20 mL, about 0.5 mL to about 15 mL, or about 1 mL to about 10 mL. In some embodiments, the solution is administered or delivered in an amount (“a dose”) of about 1.0 mL, about 1.5 mL, about 2.0 mL, about 2.5 mL, about 3.0 mL, about 3.5 mL, about 4.0 mL, about 4.5 mL, about 5.0 mL, about 5.5 mL, about 6.0 mL, about 6.5 mL, about 7.0 mL, about 7.5 mL, about 8.0 mL, about 8.5 mL, about 9.0 mL, about 9.5 mL, about 10.0 mL, about 10.5 mL, about 11.0 mL, about 11.5 mL, about 12.0 mL, about 12.5 mL, about 13.0 mL, about 13.5 mL, about 14.0 mL, about 14.5 mL, about 15.0 mL, about 15.5 mL, about 16.0 mL, about 16.5 mL, about 17.0 mL, about 17.5 mL, about 18.0 mL, about 18.5 mL, about 19.0 mL, about 19.5 mL, or about 20.0 mL.

In some embodiments, the method comprises administering or delivering a solution comprising an alkalinized buffer to the airway of a subject, wherein the alkalinized buffer comprises a buffer. In some embodiments, the buffer is provided in a dose in an amount ranging from about 10.0 mg to about 40.0 mg, about 15.0 mg to about 35.0 mg, or about 20.0 mg to about 30.0 mg. In some embodiments, the buffer is provided in the dose in an amount of about 15.0 mg, about 15.5 mg, about 20.0 mg, about 20.5 mg, about 21.0 mg, about 21.5 mg, about 22.0 mg, about 22.5 mg, about 23.0 mg, about 23.5 mg, about 24 mg, about 24.5 mg, about 25.0 mg, about 25.5 mg, about 26.0 mg, about 26.5 mg, about 27.0 mg, about 27.5 mg, about 28.0 mg, about 28.5 mg, about 29.0 mg, about 29.5 mg, or about 30.0 mg. In some embodiments, the buffer is provided in a dose in an amount of 22.5 mg.

In some embodiments, the buffer is provided at a concentration of about 1.0 mg/mL to about 30.0 mg/mL, about 1.53 mg/mL to about 27 mg/mL, about 1.50 mg/mL to about 25 mg/mL, about 2 mg/mL to about 28 mg/mL, about 5 mg/mL to about 25 mg/mL, about 8 mg/mL to about 22 mg/mL about 10 mg/mL to about 20 mg/mL, or about 5 mg/mL to about 15 mg/mL. In some embodiments, the buffer is provided at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL. In some embodiments, the buffer is provided in a concentration of 9 mg/mL.

In some embodiments, the method comprises administering or delivering a solution comprising an alkalinized buffer to the airway of a subject, wherein the alkalinized buffer comprises a titrating base. In some embodiments, the titrating base is provided in the dose in an amount ranging from 1.0 mg to about 20.0 mg, about 5.0 mg to about 15.0 mg, or about 7.0 mg to about 12.0 mg. In some embodiments, the titrating base is provided in the dose in an amount of about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 10.0 mg, about 10.5 mg, or about 11.0 mg. In some embodiments, the titrating base is provided in a dose in an amount of 7.0 mg.

In some embodiments, the titrating base is present at a concentration of about 0.001 mg/mL to about 10 mg/mL, about 0.1 mg/mL to about 8 mg/mL, or about 0.5 mg/mL to about 5 mg/mL. In some embodiments, the titrating base is present in an amount of about 0.001 mg/mL, about 0.1 mg/mL, about 0.5 mg/mL, about 1 mg/mL, about 1.5 mg/mL, about 2.0 mg/mL, about 2.5 mg/mL, about 3.0 mg/mL, about 3.5 mg/mL, about 4.0 mg/mL, about 4.5 mg/mL, or about 5.0 mg/mL. In some embodiments, the titrating base is present at a concentration of about 2.5 mg/mL. In some embodiments, the titrating base is present at a concentration of 2.5 mg/mL.

In some embodiments, the method comprises administering or delivering a solution comprising an alkalinized buffer to the airway of a subject, wherein the alkalinized buffer comprises an osmotic balancing agent. In some embodiments, the titrating base is provided in the dose in an amount ranging from 5 mg to about 30 mg, about 10 mg to about 25 mg, or about 15 mg to about 20 mg. In some embodiments, the titrating base is provided in the dose in an amount of about 10 mg, about 10.5 mg, about 11.0 mg, about 11.5 mg, about 12.0 mg, 12.5 mg, about 13.0 mg, about 13.5 mg, about 14.0 mg, about 14.5 mg, about 15.0 mg, about 15.5 mg, about 16.0 mg, about 16.5 mg, about 17.0 mg, about 17.5 mg, about 18.0 mg, about 18.5 mg, about 19.0 mg, about 19.5 mg, or about 20.0 mg. In some embodiments, the osmotic balancing agent is present in a dose in an amount of 14.0 mg.

In some embodiments, the osmotic balancing agent is present at a concentration of about 1 mg/mL to about 20 mg/mL, about 2 mg/mL to about 15 mg/mL, or about 3 mg/mL to about 10 mg/mL. In some embodiments, the osmotic balancing agent is provided at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL. In some embodiments, osmotic balancing agent is present at a concentration of about 5 mg/mL to about 6 mg/mL. In some embodiments, the osmotic balancing agent is present at a concentration of about 5.6 mg/mL. In some embodiments, the osmotic balancing agent is present at a concentration of 5.6 mg/mL.

In an illustrative embodiment, the solution comprises an alkalinized buffer, wherein the alkalinized buffer comprises a buffer in an amount of about 20.0 mg to about 25.0 mg, a titrating base in an amount of about 5.0 mg to about 10.0 mg, and an osmotic balancing agent in an amount of about 12.0 mg to about 18.0 mg. In some embodiments, the buffer is present in an amount of about 22.5 mg. In some embodiments, the titrating base is present in an amount of about 7 mg. In some embodiments, the osmotic balancing agent is present in an amount of about 14 mg.

In some embodiments, the dose comprises a concentration of about 17 mM to about 300 mM (about 1.53 mg/mL to about 27 mg/mL) of the buffer in the solution.

In some embodiments, wherein an anti-viral composition is administered, the antiviral composition may be administered close in time to the administration or delivery of the solution. In some embodiments, wherein an anti-viral composition is administered, the antiviral composition may be administered spaced apart in time to the administration or delivery of the solution, such as about 15 minutes, about 20 minutes, about 25 minutes, about 30 minutes, about 35 minutes, about 40 minutes, about 45 minutes, about 50 minutes, about 55 minutes, about 60 minutes, or about 1.25 hours, about 1.5 hours, about 1.75 hours, about 2.0 hours, about 2.25 hours, about 2.5 hour, about 2.75 hours, about 3.0 hours, or about 12 hours, about 18 hours, about 24 hours, about 36 hours, about 48 hours, about 72 hours, or about 60 hours. In some embodiments, the antiviral is administered before administering or delivering the solution. In some embodiments, the antiviral is administered after administering or delivering the solution. In some embodiments, the antiviral is co-administered with the administering or delivering of the solution. Co-administering refers to providing at least compositions at the same time or very near at the same time. The therapeutically effective amount of the antiviral composition can be determined by one of ordinary skill in the art such as a doctor nurse practitioner, nurse, pharmacist, pharmacist technician and the like.

In some embodiments, wherein a serine protease inhibitor is administered or delivered, the serine protease inhibitor is administered or delivered close in time to the solution. In some embodiments, wherein the serine protease inhibitor is administered, the serine protease inhibitor may be administered spaced apart in time to the administration or delivery of the solution, such as about 15 minutes, about 20 minutes, about 25 minutes, about 30 minutes, about 35 minutes, about 40 minutes, about 45 minutes, about 50 minutes, about 55 minutes, about 60 minutes, or about 1.25 hours, about 1.5 hours, about 1.75 hours, about 2.0 hours, about 2.25 hours, about 2.5 hour, about 2.75 hours, about 3.0 hours, or about 12 hours, about 18 hours, about 24 hours, about 36 hours, about 48 hours, about 72 hours, or about 60 hours. In some embodiments, the serine protease inhibitor is administered or delivered before administering or delivering the solution. In some embodiments, the serine protease inhibitor is administered or delivered after administering or delivering the solution. In some embodiments, the serine protease inhibitor is co-administered with the administering or delivering of the solution. The therapeutically effective amount of the antiviral composition can be determined by one of ordinary skill in the art such as a doctor nurse practitioner, nurse, pharmacist, pharmacist technician and the like.

In some embodiments, the solution is administered or delivered in a dose, and wherein the dose is provided in a dosing regimen of at least once a day, twice a day, or up to three times a day. In some embodiments, the dosing regimen of at least once a day, twice a day, or up to three times a day, may occur every day, every other day, every two days, every three days, every four days, every five days, every six days, or once a week. In some embodiments, the dosing regimen may last for at least one day, at least two days, at least three days, at least four days, at least five days, at least six days, or at least seven days. The dosing regimen may last for 10 days, 14 days, 18 days, 22 days, 26 days, or 28 days. The dosing regimen may last for about 1 week, about 2 weeks, about 3 weeks, about 4 weeks, about 5 weeks about 6 weeks, about 7 weeks, or about 8 weeks. In some embodiments, the dosing regimen may be a chronic therapy for a subject, wherein the dosing regimen is for the rest of the subject's life.

In some embodiments, the administered or delivered solution is capable of raising the airway pH to between about 8.0 to about 10.5. In some embodiments, the solution is capable of raising the pH of the epithelial cells to about 8.0 to about 11.5, about 8.5 to about 11.0, about 9.0 to about 10.5, about 9.5 to about 10.0. In some embodiments, the subjects exhaled breath condensate has a pH of about 9.0, about 9.1, about 9.2, about 9.3, about 9.4, about 9.5, about 9.6, about 9.7, about 9.8, about 9.9, about 10.0, about 10.1, or about 10.2 after administering, delivering, or contacting the airway with the solution.

In some embodiments, the viral infection rate in the subject's epithelial cells is reduced by about 85% to about 100% as compared to a subject who has not received the solution.

In some embodiments, the subject has been diagnosed with a respiratory viral infection such as COVID-19. In some embodiments, the subject is suspected of having a viral infection, such as COVID-19. In some embodiments, the solution may be delivered to a subject's airways in an effort to reduce the chance of contracting a viral respiratory infection or reduce the viral respiratory infection's impact on the subject's airways.

In some embodiments, the solution further comprises a scented composition configured to provide a pleasant scent. In some embodiments, the scented composition is an essential oil. In some embodiments, the scented composition is an herb, flower such as chamomile, or perfume. In some embodiments, the scented composition is present in an amount of about 0.001% to about 10.0% by volume of the total solution volume. In some embodiments, the scented composition is present in an amount of about 0.001%, about 0.005%, about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by volume of the total solution volume.

Alleviating a symptom refers to reducing the discomfort caused by a symptom associated with a respiratory viral infection including inflammation, labored breathing, severe cough, and reduced lung function.

Treating refers to killing or inhibiting viral replication, reducing or preventing viral entry into an airway cell, or reducing or alleviating a symptom associated with a viral respiratory infection.

Preventing or reducing viral entry refers to inhibiting the CatB/L pathway to block virus entry into an airway cell. By preventing or reducing viral entry, the viral load with airway cells will be lower compared to a subject who has not received treatment. This allows the subjects own immune system a better chance at overcoming the viral infection.

Subject refers to any animal but preferably a human. The method is not limited to a human of a certain age but rather is applicable to all ages.

In some embodiments, a kit is provided. In some embodiments, the kit comprises a solution comprising an alkalinized buffer. In some embodiments, the kit comprises a container including the solution and a delivery device. In some embodiments, the solution further comprises a scented composition. In some embodiments, the solution is prepackaged in the delivery device. In some embodiments, the kit includes a delivery device comprising the solution and a scented composition configured to have a pleasant scent. In some embodiments, the kit further comprises packaging. In some embodiments, the kit further comprises instructions for use. In some embodiments, the kit comprises a container, a delivery device, a solution and a scented composition. In some embodiments, the kit further comprises an antiviral composition or a serine protease inhibitor. In some embodiments, the kit comprises replacement cartridges comprising the solution for loading into a delivery device. In some embodiments, the cartridges further comprise a scented composition.

### EXAMPLES AND METHODOLOGY

### Example 1: The Solution Increased pH Intracellularly in Human Airway Epithelial Cells

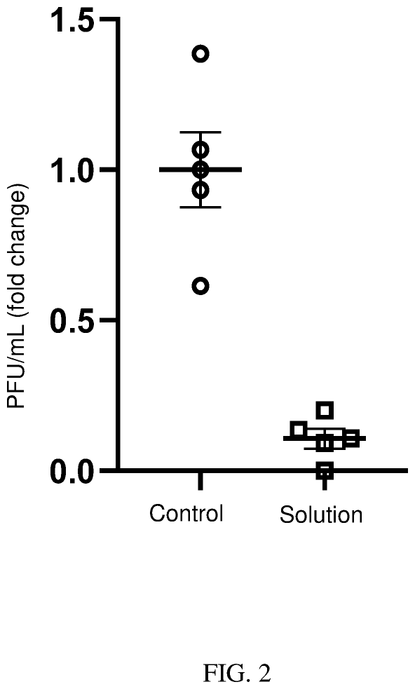

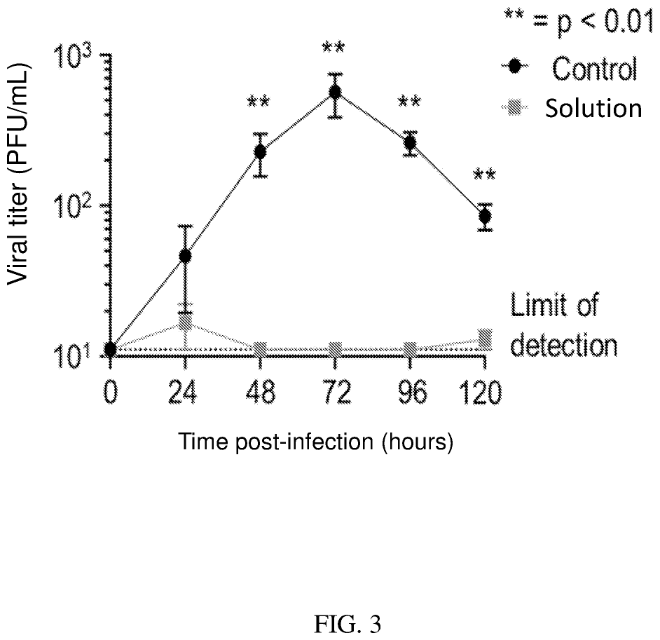

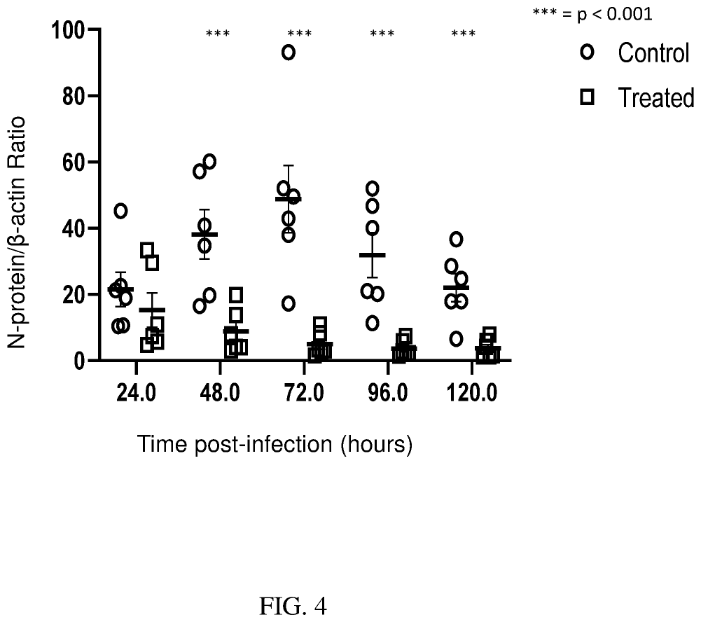

Primary normal human airway epithelial (HAE) cells from both upper and lower airways were collected and cultured. In the Examples provided below, the HAE cells were grown at air liquid interface (ALI) to evaluate the solution's ability to raise intracellular pH. Pretreatment and post-infection treatments were evaluated in HAE cells grown in submerged cultures to evaluate the solution's ability to prevent SARS-CoV-2 replication and viral infection rate in HAE cells (FIGS. 2-5).

As will be discussed in further detail below, the glycine-mediated intracellular alkalinization inhibited SARS-CoV-2 replication by the CatB/L activation pathway. An exemplary embodiment of the solution was prepared as shown in Table 1 and used to raise intracellular pH in cultured HAE cells obtained from normal subjects.

TABLE 1
Exemplary formulation of a 2.5 mL solution

| | mol | Dose | conc. | Final conc. |
|---|---|---|---|---|
| Components | wt. | (mg) | mg/ml | (mM) |
| Glycine | 75 | 22.5 | 9 | 120 |
| NaCl | 55 | 14 | 5.6 | 102 |
| NaOH | 40 | 7 | 2.8 | 70 |

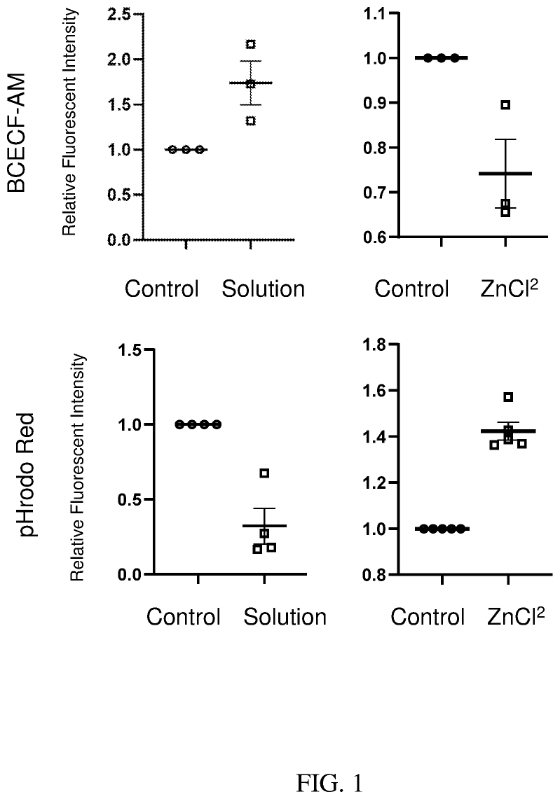

In a first experiment, the cells were pre-loaded for about ten minutes with 1.25 μM 2′,7′-bis-(2-carboxyethyl)-5-(and-6)-carboxyfluorescein, acetoxymethyl ester (BCECF-AM, ThermoFisher Scientific, USA), a pH-sensitive dye that increases in fluorescence intensity as pH increases. The HAE cells were then imaged with fluorescence microscopy (EVOS M5000, GFP light cube, excitation 470/emission 525, ThermoFisher Scientific, USA) before and after contact with the solution prepared pursuant to Table 1. Data was normalized to pre-treatment fluorescence intensity. Fluorescence intensity increased significantly in three wells from two normal subjections (n=3, p.s. 0.05). As a control, cells from the same subjects were treated with ZnCl₂ (100 μM) which acidifies the intracellular space by inhibiting Hv1. Zinc treatment had an effect opposite that of the solution, essentially eliminating fluorescence (FIG. 1, n=3; p<0.05), as shown in FIG. 1. FIG. 1 shows that pH increased significantly. Cells tolerated this treatment well, with no change in viability as assessed by Trypan blue (not shown). This finding was confirmed with a second pH-sensitive dye, 20 μpHrodo Red (ThermoFisher Scientific, USA), which decreases in fluorescence intensity (excitation 540 nm; emission 593 nm) as pH increases (FIG. 1). As a negative control, ZnCl₂ (100 μM) decreased BCECF-AM fluorescence by inhibiting Hv1 (FIG. 1). Hv1 is expressed at the apex of primary HAE cells, where it complements loss of CFTR (keeping protons intracellularly when there is decreased bicarbonate secretion). It is notable that there are no reports of patients with cystic fibrosis succumbing to COVID-19, making it conceivable that intracellular retention of bicarbonate is protective in these patients.

### Example 2: Previous Studies Show an Alkalinized Buffer Solution Formulated as an Aerosol is Safe for Humans to Breathe

Previous studies, looking at asthma, found that an alkalinized buffer composition was safe for human inhalation. Nebulization of the solution for inhalation by humans is safe for normal individuals and patients with chronic lung disease (See Davis, M. D., et al. “Safety of an alkalinizing buffer designed for inhaled medications in humans,” Clinical Trail, Respir. Care, 2013, 58(7) 1226-32 the contents of which are incorporated herein by reference.) Airway alkalization markedly increased exhaled breath pH and decreased exhaled nitric oxide levels. Airway acidification plays a role in disorders of the pulmonary tract. The inhalation of the alkalinized buffer composition measurably alkalinized the airways without compromising lung function or causing adverse events.

### Example 3: Airway Acidification is an Indication of Inflammation and an Accepted Method of Analyzing and Monitoring Respiratory Diseases' Response to Therapeutics

Regulation of airway pH plays a role in the pathogenesis of obstructive lung diseases. Airway acidification, caused by both intrinsic and extrinsic factors, is associated with neutrophilic and eosinophilic inflammation, bronchospasm, bronchial hyper-reactivity, ciliary dysfunction, epithelial dysfunction, augmented oxidative damage, abnormal fluid transport, inhibition of transport of cationic drugs such as albuterol, and alteration of cellular death pathways, including inhibition of apoptosis. Improved ability to treat or potentially reverse acidic airway pathology by therapeutic alteration of airway pH impacts respiratory medicine. The ability to normalize airway pH via inhalation allows introduction of new pulmonary therapeutics.

Airway lining fluid (ALF) acidity can be qualitatively determined noninvasively via the collection of exhaled breath condensate (EBC) and measuring its pH. Assays to measure EBC pH have been developed for patients of all ages and sizes, including those receiving mechanical ventilation. EBC pH normally is within a mildly alkaline range of pH 7.5 to pH 8.2. EBC has minimal buffer capacity, which allows EBC to assess the presence of volatile acids in ALF, indicated by a change in EBC pH. Although a normal EBC pH does not exclude airway acidity at some level, a low EBC pH value is highly specific for acidity somewhere within the airway.

Using EBC methods, studies have shown that patients with COPD, asthma bronchiectasis, cystic fibrosis, and chronic cough have airway acidification. EBC pH in this intervention was used as a noninvasive safety measure to assess any excessive alkalinization.

Airway pH also affects fraction of exhaled nitric oxide (FeNO) levels by simple chemistry. As the ALF pH decreases, commonly present nitrite becomes protonated to nitrous acid, which decomposes to nitric oxide which is then in part exhaled. FeNO analyzers can qualitatively assess the alkalinizing effects of alkaline inhalation therapy by monitoring decreased levels of FeNO.

### Example 4: Evaluating the Solution's Ability to Reduce Viral Infection

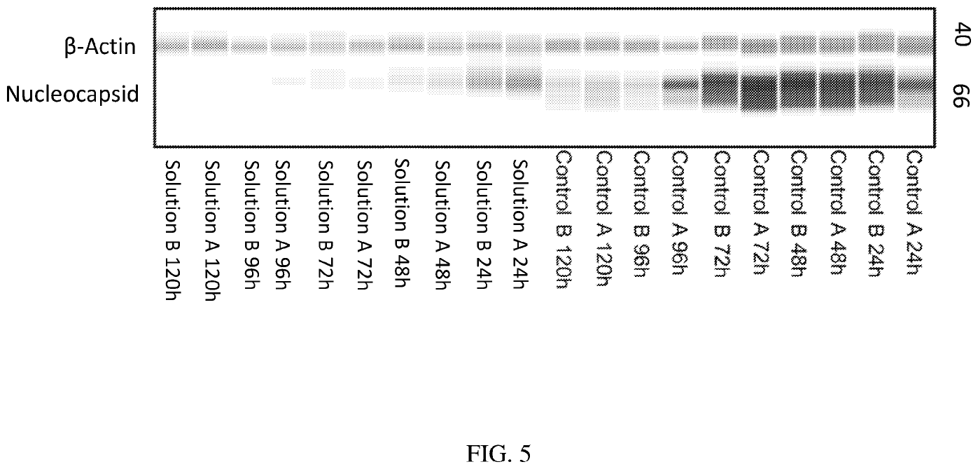

Therapeutic use of the solution is evaluated first by determining if alkalinization of airway epithelial cells inhibits SARS-CoV-2 infection. Cells harvested and maintained in a core facility are grown from normal human airway epithelium (HAE). Two donors provided HAE cells for in vitro analysis and identified as Group A and Group B (as seen in FIG. 5). For speed and imaging ease, cells are used when epithelial cells have completely replaced irradiated fibroblasts and are confluent, but they are not grown at air liquid interface cell culture (ALI) which takes 4-6 weeks per culture. Cells are treated with or without the solution (120 mM glycine buffer).

After dosing the cells as described above, the cells were then exposed apically to SARS-CoV-2 (grown in Vero-E6 cells) at an MOI of 1.0 for two hours at 37° C. After absorption, cells are washed once with PBS, and then the inoculum is replaced with fresh culture media (DMEM supplemented with 10% FBS, 100 units/ml penicillin, and 100 mg/ml streptomycin). As the results show, the solution inhibited viral cell entry and replication in HAE cells compared to the non-treated HAE cells as shown in FIGS. 2, 3, 4, (labeled control) and 5 (labeled control A and control B).

### Example 5: The Solution has an Anti-Viral Effect

Each experiment requires three replicates of two wells each, each of the three from a different human donor so in total 16 six-well plates. At 20 hours post-infection, cells were lysed in RIPA buffer. Lysates were collected and sterilized with UV light in the treatment wells and placed into blinded, bar-coded Eppendorf tubes. These tubes were re-sterilized by UV light and washed externally with bleach, and then analyzed for SARS CoV-2 S1 and N proteins (Sino Biological) using antibodies and the quantitative multiplexed Jess capillary immunoblot system (ProteinSimple). Ratio of the 180 kDa unprocessed S protein to the cleaved S2 subunit of the S protein was compared between treatments. For statistical analysis, differences in mean ratios were compared by ANOVA.

Both the solution and CM inhibit cleavage relative to untreated cells, and this effect is augmented by additive use, supporting the solution as a therapy, either alone or in conjunction with a serine protease inhibitor. The serine protease inhibitor could be any of those known to one skilled in the art, the efficacy of which would be enhanced by the solution.

There could be differences between donors; given the detailed information about gene and protein expression in these donors, knowledge of which donors are more resistant is of interest. Compensatory upregulation of proteins such as carbonic anhydrase (CA) 2 occurs with epithelial solution exposure. In some embodiments, the method further comprises administering or delivering a CA or other inhibitors such as acetazolamide to the subject's airways. The solution in combination with CM could have benefits in addition to preventing S protein cleavage. Alveolar Type II cells could be the primary target, though other airway cells appear to be targets.

The Vero-E6 cells are washed and lysed, assaying for viral entry (viral RNA) by PCR. Briefly, after infection, culture supernatants are collected and viral RNA is extracted. Viral genomes are quantified by qRT-PCR using primers designed for the viral polymerase, E, or N genes. Thirty-two six-well plates are used.

HAE cells plaque-forming units and intracellular viral RNA is inhibited in rank order as follows: solution+MC>solution alone>control, suggesting that solution has anti-viral effect.

In one embodiment, a therapeutic regimen of the solution delivered by ultrasonic or conventional nebulizer, 3 ml, every 1-6 hours depending on symptoms. There is increasing efficacy with increasing dose and time that does not affect cell viability. This information informs patient regimens including dose and frequency. Inhaled solution prevents SARS-CoV-2 entry into airway epithelial cells. This benefits patients with SARS-CoV-2 infection both by preventing virus spread and decreasing airway injury.

While the concepts of the present disclosure are susceptible to various modifications and alternative forms, specific exemplary embodiments of the disclosure have been shown by way of example in the drawings. It should be understood, however, that there is no intent to limit the concepts of the present disclosure to the particular disclosed forms; the intention is to cover all modifications, equivalents, and alternatives falling within the spirit and scope of the invention as defined by the claims. Although this disclosure refers to specific embodiments, it will be understood by those skilled in the art that various changes in form and detail may be made without departing from the subject matter set forth in the accompanying claims.

1. A method of treating a viral respiratory infection caused by a SARS-CoV-2 virus comprising:
a) contacting the airways of a subject with a solution, the solution comprising an alkalinized buffer comprising i) a buffer, ii) a titrating base, and iii) an osmotic balancing agent,
wherein the solution has a pH ranging from about 7.4 to about 11.5, and wherein the solution is formulated as an aerosol; and
b) contacting the airways with a second composition comprising an antiviral or serine protease inhibitor.

2. The method of claim 1, wherein the solution inhibits the CatL/B pathway in the airway epithelial cells and reduces the viral infection rate.

3. (canceled)

4. The method of claim 1, wherein the second composition comprises remdesivir.

5. The method of claim 1, wherein the buffer comprises an amino acid selected from glycine, alanine, valine, leucine, isoleucine, methionine, tryptophan, asparagine, glutamine, serine, threonine, tyrosine, cysteine, aspartic acid, glutamic acid, arginine, or histidine.

6. (canceled)

7. The method of claim 5, wherein the buffer comprises glycine and is present at a concentration of about 50 mM to 250 mM, and has a pH ranging from about 8 to about 11.5.

8. (canceled)

9. The method of claim 1, wherein the titrating base comprises a hydroxide and is present at a concentration ranging from about 25 mM to about 150 mM.

10. The method of claim 9, wherein the titrating base has a pH ranging from about 9 to about 14.

11. The method of claim 1, wherein the osmotic balancing agent comprises sodium and is present at a concentration of about 50 mM to about 200 mM.

12. The method of claim 1, wherein the alkalinized buffer further comprises an aqueous solution safe for inhalation.

13. (canceled)

14. The method of claim 1 further comprising the step of delivering the solution to make contact with the subject's airways using a delivery device selected from a nebulizer, a nasal spray bottle, a squeeze bottle, or a solution atomizer, optionally wherein the nebulizer is selected from an ultrasonic nebulizer, conventional nebulizer, or vibrating mesh nebulizer.

15. The method of claim 14, wherein the solution is delivered at temperature ranging from about 18° C. to about 40° C.

16. (canceled)

17. The method of claim 1, wherein the solution is provided in an amount ranging about 0.25 mL to about 10 mL.

18. The method of claim 7, wherein the buffer is present in the solution in an amount of about 120 mM.

19. The method of claim 2, wherein the second composition is a serine protease inhibitor, optionally wherein the serine protease inhibitor is camostat methylate.

20. (canceled)

21. A method of treating a viral respiratory infection in a subject in need thereof, comprising administering to the subject an aqueous alkaline solution consisting of a buffer, a titrating base, and an osmotic balancing agent, wherein the solution is designed for inhalation or nasal administration, thereby treating the subject.

22. The method of claim 21, wherein the buffer comprises an amino acid selected from the group consisting of glycine, alanine, valine, leucine, isoleucine, methionine, tryptophan, asparagine, glutamine, serine, threonine, tyrosine, cysteine, aspartic acid, glutamic acid, arginine and histidine.

23. The method of claim 21, wherein the titrating base is sodium hydroxide, and the osmotic balancing agent is sodium chloride.

24. (canceled)

25. The method of claim 21, wherein the solution has a pH of about 8 to 10, optionally wherein the pH is about 9.4 to about 9.8.

26. (canceled)

27. The method of claim 21, wherein the concentration of the buffer in the solution is from about 17 mM to about 300 mM (from about 1.53 to about 27 mg/mL).

28. The method of claim 21, wherein the solution is administered to the subject at a dose from about 1 mg/ml to about 27 mg/ml.

29. The method of claim 21, further comprising administering a serine protease inhibitor or an antiviral, optionally wherein the antiviral is remdesivir.

30. (canceled)

31. (canceled)
